(19) United States
(12) Patent Application Publication
(10) Pub. No.: US 2024/0285464 A1
(43) Pub. Date: Aug. 29, 2024

(54) QUADRANTANOPIAL OCCLUSIVE LENSES AND METHOD OF USE

(71) Applicant: Rosenthal Holdings, LLC, Carrollton, TX (US)

(72) Inventor: David Rosenthal, Carrollton, TX (US)

(73) Assignee: Rosenthal Holdings, LLC, Carrollton, TX (US)

(21) Appl. No.: 18/655,775

(22) Filed: May 6, 2024

Related U.S. Application Data
(63) Continuation-in-part of application No. 18/109,302, filed on Feb. 14, 2023.
(60) Provisional application No. 63/311,097, filed on Feb. 17, 2022.

(51) Int. Cl. A61H 5/00; A61M 21/00

(52) U.S. Cl. CPC A61H 5/00; A61M 21/00; A61M 2021/0044; A61M 2205/056

(57) ABSTRACT

Quadrantanopial occlusive lenses and method of use includes the steps of performing a vision zone assessment test to identify a field area of a patient's field of view having a selected characteristic, based on the identified field area, providing an appliance having a non-transparent optical characteristic associated with the field area, the patient wearing the appliance, and while the patient is wearing the appliance, the patient performing a selected activity associated with the neurological performance. The neurological performance may be associated with the quality of performance of the selected activity. The selected activity may be a physical activity. The step of performing a vision zone assessment test may include moving a visual target about the patient's field of view. The step of performing a vision zone assessment test may include observing the patient's eye movements in response to a moving visual stimulus.

### CROSS-REFERENCE TO RELATED APPLICATION

This application is a Continuation-in-Part of U.S. patent application Ser. No. 18/109,302 filed on Feb. 14, 2023, entitled “QUANDRANTNOPIAL OCCLUSIVE LENSES,” which claims the benefit of U.S. Provisional Patent Application No. 63/311,097 filed on Feb. 17, 2022, entitled “DR QUANDRANTNOPIAL OCCLUSIVE LENSES,” which are hereby incorporated by reference in their entirety for all that is taught and disclosed therein.

### FIELD OF THE INVENTION

The present invention relates to lenses, and more particularly to quadrantanopial occlusive lenses and method of use that enable selective occlusion of the visual system of a patient to improve neurological performance.

### BACKGROUND AND SUMMARY OF THE INVENTION

Occlusion of the visual system is sometimes needed by athletes, those who struggle with neurological diseases, and those looking to improve their overall brain performance. Occlusion limits information from a specific visual field that connects with a corresponding region of the brain. This visual information would otherwise reach the brain and create a demand on the brain to either interpret or inhibit that incoming visual information.

Therefore, a need exists for new and improved quadrantanopial occlusive lenses and method of use that enable selective occlusion of the visual system of a patient to improve neurological performance. In this regard, the various embodiments of the present invention substantially fulfill at least some of these needs. In this respect, the quadrantanopial occlusive lenses and method of use according to the present invention substantially depart from the conventional concepts and designs of the prior art, and in doing so provide an apparatus primarily developed for the purpose of enabling selective occlusion of the visual system of a patient to improve neurological performance.

The present invention provides improved quadrantanopial occlusive lenses and method of use, and overcomes the above-mentioned disadvantages and drawbacks of the prior art. As such, the general purpose of the present invention, which will be described subsequently in greater detail, is to provide improved quadrantanopial occlusive lenses and method of use that have all the advantages of the prior art mentioned above.

To attain this, the preferred embodiment of the present invention essentially comprises the steps of performing a vision zone assessment test to identify a field area of a patient's field of view having a selected characteristic, based on the identified field arca, providing an appliance having a non-transparent optical characteristic associated with the field area, the patient wearing the appliance, and while the patient is wearing the appliance, the patient performing a selected activity associated with the neurological performance. The neurological performance may be associated with the quality of performance of the selected activity. The selected activity may be a physical activity. The step of performing a vision zone assessment test may include moving a visual target about the patient's field of view. The step of performing a vision zone assessment test may include observing the patient's eye movements in response to a moving visual stimulus. There are, of course, additional features of the invention that will be described hereinafter and which will form the subject matter of the claims attached.

There has thus been outlined, rather broadly, the more important features of the invention in order that the detailed description thereof that follows may be better understood and in order that the present contribution to the art may be better appreciated.

### BRIEF DESCRIPTION OF THE DRAWINGS

FIG. 1 is a front view of the current embodiment of quadrantanopial occlusive lenses constructed in accordance with the principles of the present invention.

FIG. 2 is a front view of a first alternative embodiment of quadrantanopial occlusive lenses constructed in accordance with the principles of the present invention.

FIG. 3 is a front view of a second alternative embodiment of quadrantanopial occlusive lenses constructed in accordance with the principles of the present invention.

FIG. 4 is a front view of a third alternative embodiment of quadrantanopial occlusive lenses constructed in accordance with the principles of the present invention.

FIG. 5 is a front view of a fourth alternative embodiment of quadrantanopial occlusive lenses constructed in accordance with the principles of the present invention.

FIG. 6 is a front view of a fifth alternative embodiment of quadrantanopial occlusive lenses constructed in accordance with the principles of the present invention.

FIG. 7 is a front view of a sixth alternative embodiment of quadrantanopial occlusive lenses constructed in accordance with the principles of the present invention.

FIG. 8 is a front view of a seventh alternative embodiment of quadrantanopial occlusive lenses constructed in accordance with the principles of the present invention.

FIG. 9 is a front view of an eighth alternative embodiment of quadrantanopial occlusive lenses constructed in accordance with the principles of the present invention.

FIG. 10 is a front view of a ninth alternative embodiment of quadrantanopial occlusive lenses constructed in accordance with the principles of the present invention.

FIG. 11 is a front view of a tenth alternative embodiment of quadrantanopial occlusive lenses constructed in accordance with the principles of the present invention.

FIG. 12 is a front view of an eleventh alternative embodiment of quadrantanopial occlusive lenses constructed in accordance with the principles of the present invention.

FIG. 13 is a flowchart of the method of use of the of quadrantanopial occlusive lenses of FIGS. 1-12.

The same reference numerals refer to the same parts throughout the various figures.

### DESCRIPTION OF THE CURRENT EMBODIMENT

An embodiment of the quadrantanopial occlusive lenses of the present invention is shown and generally designated by the reference numeral 10.

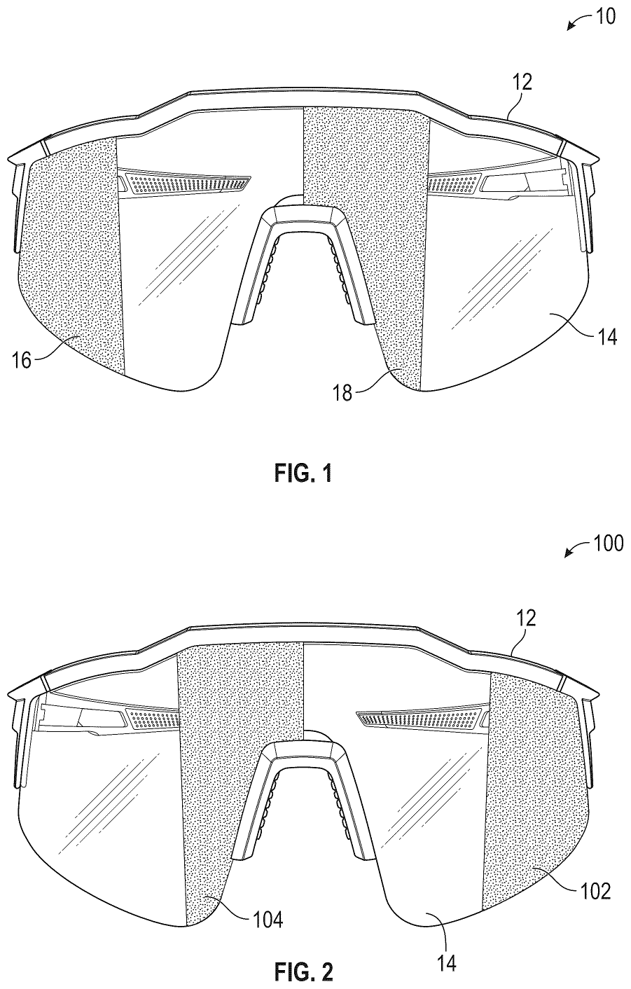

FIG. 1 illustrates the improved quadrantanopial occlusive lenses 10 of the present invention. More particularly, the quadrantanopial occlusive lenses are eyewear having a frame 12 and a lens 14. FIG. 1 shows the quadrantanopial occlusive lenses configured in a manner believed to limit the visual input going to the networks associated with the right brain (the “no networks”). Black occlusive laser etching 16 covers the outside lateral portion of vision closest to the car (the temporal field) of the wearer's right eye. Black occlusive laser etching 18 also covers the wearer's left eye closest to the nose (the nasal field).

FIG. 2 illustrates a first alternative embodiment of the improved quadrantanopial occlusive lenses 100 of the present invention. More particularly, the quadrantanopial occlusive lenses are eyewear having a frame 12 and a lens 14. FIG. 2 shows the quadrantanopial occlusive lenses configured in a manner believed to limit the visual input going to the networks associated with the left brain (the “yes networks”). Black occlusive laser etching 102 covers the outside lateral portion of vision closest to the car (the temporal field) of the wearer's left eye. Black occlusive laser etching 104 also covers the wearer's right eye closest to the nose (the nasal field).

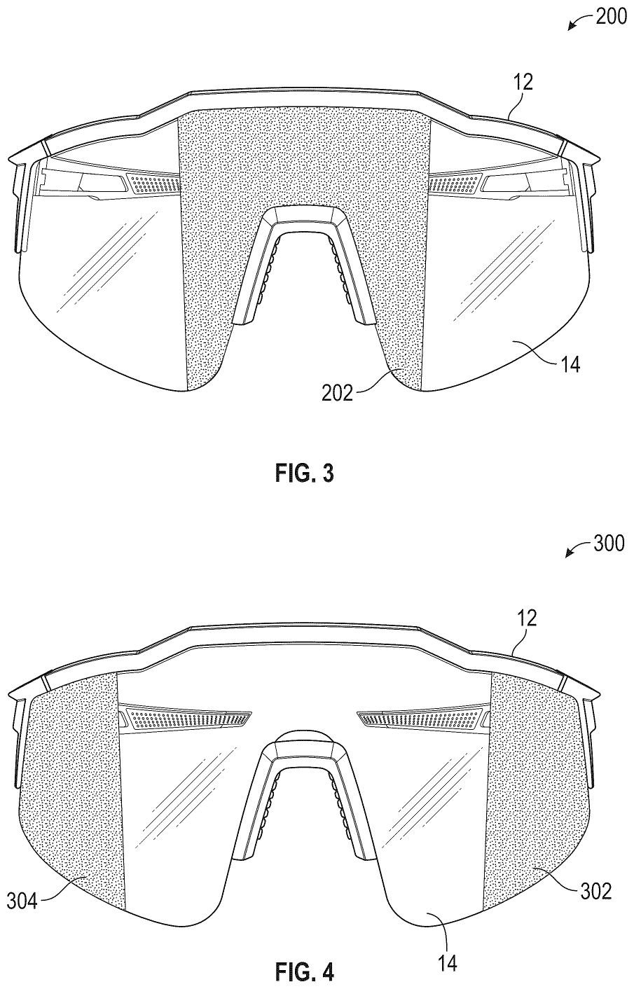

FIG. 3 illustrates a second alternative embodiment of the improved quadrantanopial occlusive lenses 200 of the present invention. More particularly, the quadrantanopial occlusive lenses are eyewear having a frame 12 and a lens 14. FIG. 3 shows the quadrantanopial occlusive lenses configured in a manner believed to limit the visual input going to the bi-nasal fields associated with portions of both the left brain and the right brain. This style of occlusion allows the wearer's eyes to look further into the distance and inhibits near vision targets. Black occlusive laser etching 202 covers the wearer's left and right eyes closest to the nose (the left and right nasal fields).

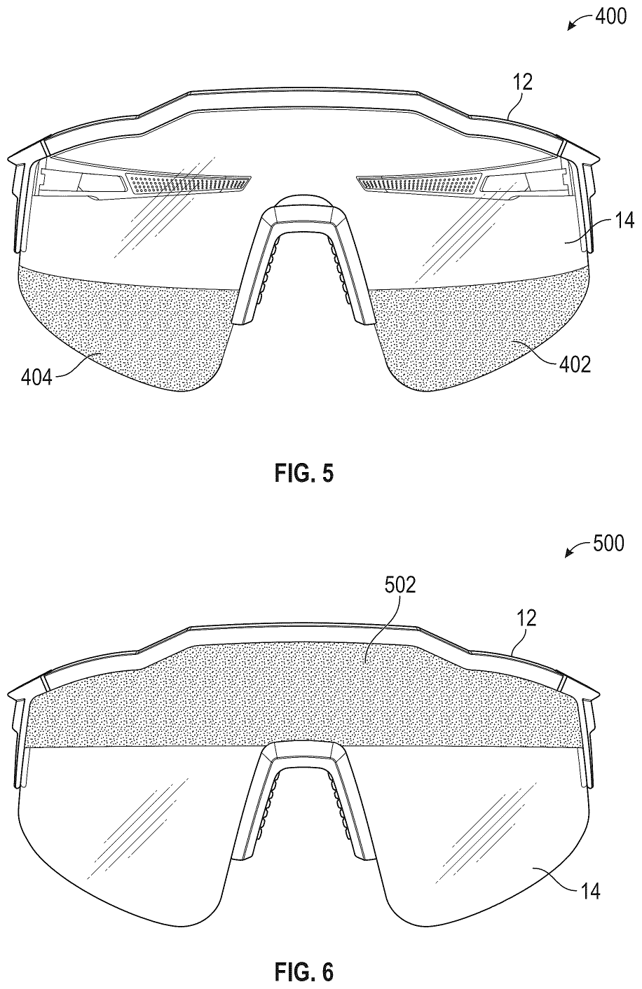

FIG. 4 illustrates a third alternative embodiment of the improved quadrantanopial occlusive lenses 300 of the present invention. More particularly, the quadrantanopial occlusive lenses are eyewear having a frame 12 and a lens 14. FIG. 4 shows the quadrantanopial occlusive lenses configured in a manner believed to limit the visual input going to the bi-temporal fields associated with portions of both the left brain and the right brain. This style of occlusion allows the wearer's eyes to focus on near vision targets and inhibits far vision targets. Black occlusive laser etching 302 covers the outside lateral portion of vision closest to the car (the temporal field) of the wearer's left eye. Black occlusive laser etching 304 covers the outside lateral portion of vision closest to the ear (the temporal field) of the wearer's right eye.

FIG. 5 illustrates a fourth alternative embodiment of the improved quadrantanopial occlusive lenses 400 of the present invention. More particularly, the quadrantanopial occlusive lenses are eyewear having a frame 12 and a lens 14. FIG. 5 shows the quadrantanopial occlusive lenses configured in a manner believed to limit the visual input coming from the inferior (lower) fields of vision associated with portions of both the left brain and the right brain parietal lobes. This style of occlusion allows the wearer's eyes to only access superior-temporal vision and inhibits inferior parietally-based targets. Black occlusive laser etching 402 covers the inferior portion of vision of the wearer's left eye. Black occlusive laser etching 404 covers the inferior portion of vision of the wearer's right eye.

FIG. 6 illustrates a fifth alternative embodiment of the improved quadrantanopial occlusive lenses 500 of the present invention. More particularly, the quadrantanopial occlusive lenses are eyewear having a frame 12 and a lens 14. FIG. 6 shows the quadrantanopial occlusive lenses configured in a manner believed to limit the visual input coming from the superior (upper) fields of vision associated with portions of both the left brain and the right brain parietal lobes. This style of occlusion allows the wearer's eyes to only access inferior-parietal vision and inhibits superior temporally-based targets. Black occlusive laser etching 502 covers the superior portion of vision of the wearer's left and right eyes.

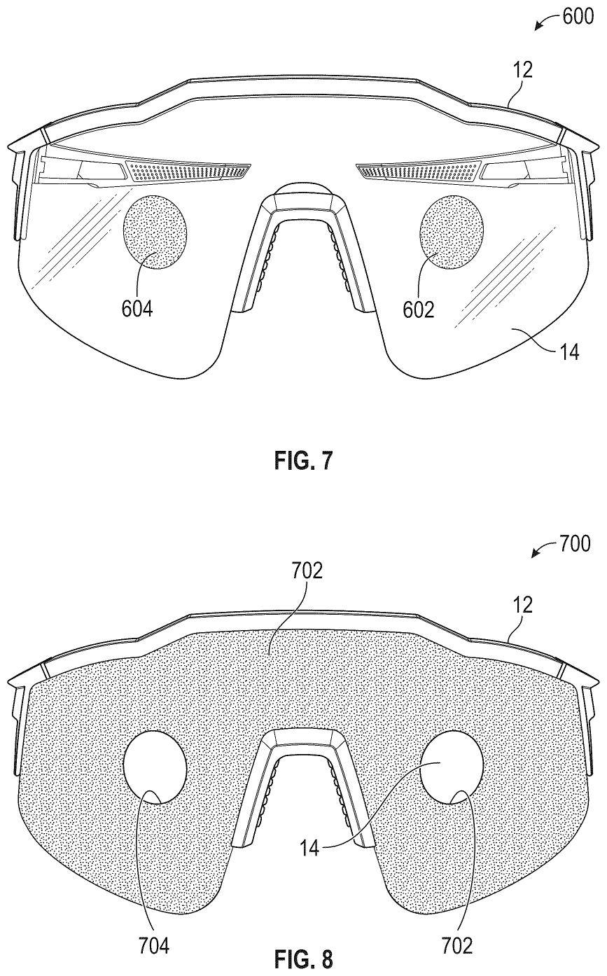

FIG. 7 illustrates a sixth alternative embodiment of the improved quadrantanopial occlusive lenses 600 of the present invention. More particularly, the quadrantanopial occlusive lenses are eyewear having a frame 12 and a lens 14. FIG. 7 shows the quadrantanopial occlusive lenses configured in a manner believed to limit the visual input coming from the central vision, which is primarily mitigated by the temporal lobes. This style of occlusion decreases color and detail information that is going directly back to the wearer's retinas. This style of occlusion also allows the wearer's eyes to only access parietally-based (peripheral) visual networks and inhibits temporally-based color and detail of visual targets. Black occlusive laser etching 602 covers the central portion of vision of the wearer's left eye. Black occlusive laser etching 604 covers the central portion of vision of the wearer's right eye.

FIG. 8 illustrates a seventh alternative embodiment of the improved quadrantanopial occlusive lenses 700 of the present invention. More particularly, the quadrantanopial occlusive lenses are eyewear having a frame 12 and a lens 14. FIG. 8 shows the quadrantanopial occlusive lenses configured in a manner believed to limit the visual input coming from the peripheral vision, which is primarily mitigated by the parietal lobes. Vision is only possible through a pair of 16 mm apertures 704, 706 in the current embodiment. This style of occlusion decreases motion and contrast-based information that is going directly back to the wearer's retinas. This style of occlusion also allows the wearer's eyes to only access temporally-based (central) visual networks and inhibits parietally-based motion and contrasting visual awareness. Black occlusive laser etching 702 covers entire lens except for the wearer's pupils.

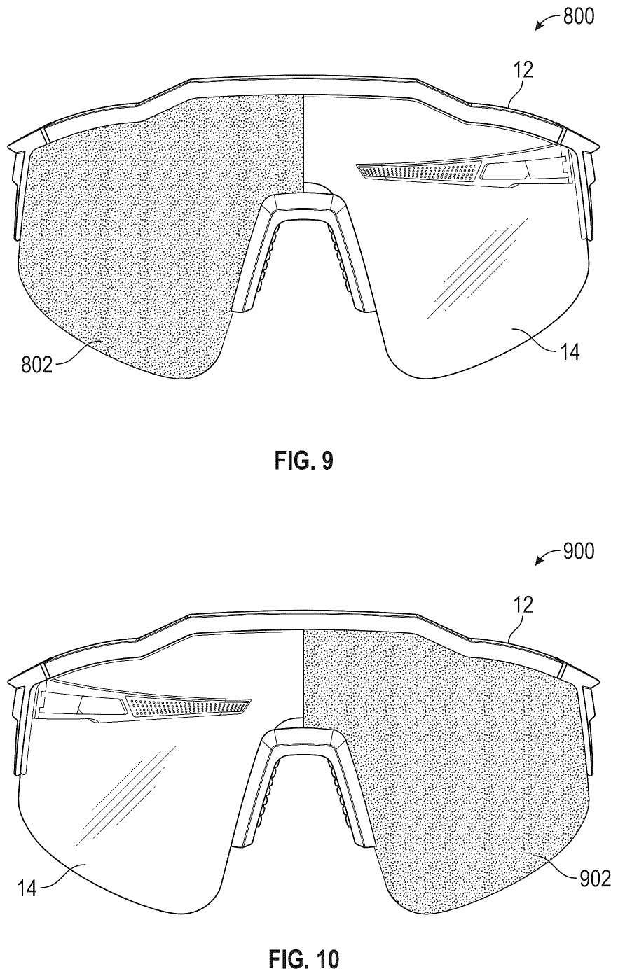

FIG. 9 illustrates an eighth alternative embodiment of the improved quadrantanopial occlusive lenses 800 of the present invention. More particularly, the quadrantanopial occlusive lenses are eyewear having a frame 12 and a lens 14. FIG. 9 shows the quadrantanopial occlusive lenses configured in a manner believed to limit the visual input coming from the nasal pathway and temporal field of the wearer's right eye. Vision is only possible through the wearer's left eye. This style of occlusion inhibits both central and peripheral information from the wearer's right eye. This style of occlusion allows activation of temporally-based (central) visual networks and parietal he-based motion and contrasting visual awareness of only the wearer's left eye. Black occlusive laser etching 802 covers the entire right half of the lens.

FIG. 10 illustrates a ninth alternative embodiment of the improved quadrantanopial occlusive lenses 900 of the present invention. More particularly, the quadrantanopial occlusive lenses are eyewear having a frame 12 and a lens 14. FIG. 10 shows the quadrantanopial occlusive lenses configured in a manner believed to limit the visual input coming from the nasal pathway and temporal field of the wearer's left eye. Vision is only possible through the wearer's right eye. This style of occlusion inhibits both central and peripheral information from the wearer's left eye. This style of occlusion allows activation of temporally-based (central) visual networks and parietal he-based motion and contrasting visual awareness of only the wearer's right eye. Black occlusive laser etching 902 covers the entire left half of the lens.

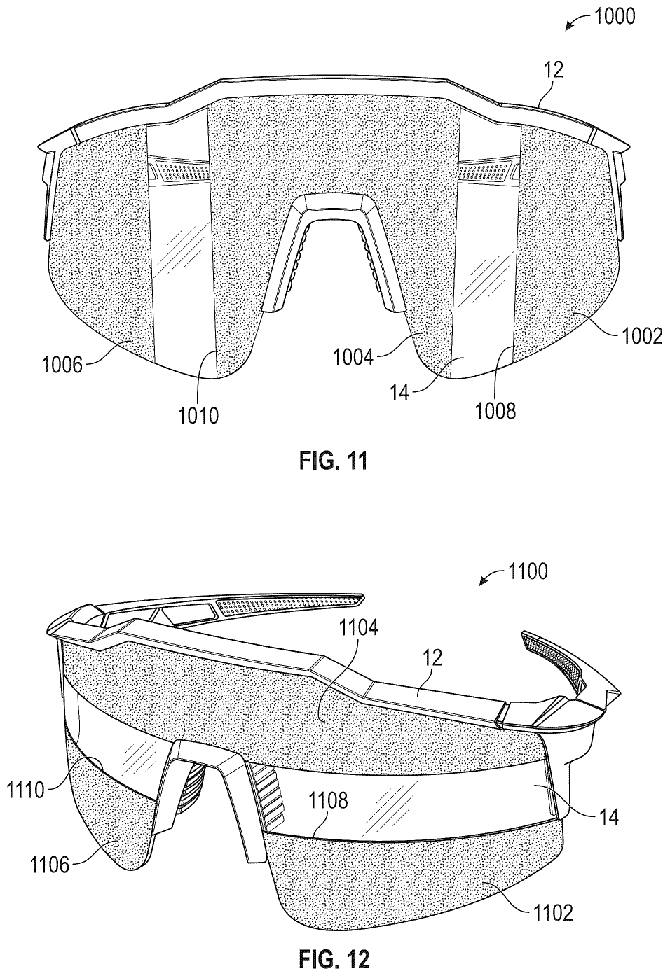

FIG. 11 illustrates a tenth alternative embodiment of the improved quadrantanopial occlusive lenses 1000 of the present invention. More particularly, the quadrantanopial occlusive lenses are eyewear having a frame 12 and a lens 14. FIG. 11 shows the quadrantanopial occlusive lenses configured in a manner believed to limit the visual input coming from the right and left eyes closest to the nose (right and left nasal fields) and the outside lateral portion of vision closest to the right and left ears (right and left temporal fields). Vision is only possible through a pair of vertical apertures 1008, 1010 in the current embodiment. This style of occlusion only allows the wearer's eyes to move up and down without losing the ability to see. Black occlusive laser etching 1002 covers the outside lateral portion of vision closest to the left ear (left temporal field). Black occlusive laser etching 1004 covers the right and left eyes closest to the nose (right and left nasal fields). Black occlusive laser etching 1006 covers the outside lateral portion of vision closest to the right car (right temporal field).

FIG. 12 illustrates a tenth alternative embodiment of the improved quadrantanopial occlusive lenses 1100 of the present invention. More particularly, the quadrantanopial occlusive lenses are eyewear having a frame 12 and a lens 14. FIG. 12 shows the quadrantanopial occlusive lenses configured in a manner believed to limit the visual input coming from the inferior (lower) and superior (upper) visual fields of the wearer's right and left eyes. Vision is only possible through a pair of horizontal apertures 1108, 1110 located in the middle of the lens in the current embodiment. This style of occlusion only allows the wearer's eyes to move left and right without losing the ability to see, which activates both the medial and lateral recti musculature. Black occlusive laser etching 1102 covers the lower third of the left half of the lens (left inferior field). Black occlusive laser etching 1104 covers the upper third of the entire lens (right and left superior fields). Black occlusive laser etching 1106 covers the lower third of the right half of the lens (right inferior field).

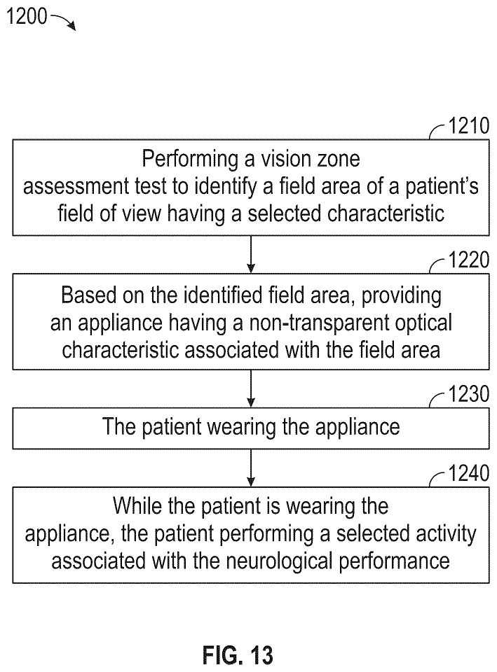

FIG. 13 illustrates the improved method of use 1200 of the quadrantanopial occlusive lenses of the present invention. More particularly, the method includes the steps of performing a vision zone assessment test to identify a field area of a patient's field of view having a selected characteristic (1210), based on the identified field area, providing an appliance having a non-transparent optical characteristic associated with the field area (1220), the patient wearing the appliance (1230), and while the patient is wearing the appliance, the patient performing a selected activity associated with the neurological performance (1240).

In the current embodiment, the neurological performance is associated with the quality of performance of the selected activity, which is can be a physical or athletic activity. The selected characteristic is abnormal eye movement when directed to a selected direction. The non-transparent optical characteristic can be opacity, translucency, or reduced transmissivity. The appliance is eyewear having a frame 12 and a lens 14.

The step of performing a vision zone assessment test (1210) can include moving a visual target about the patient's field of view. The step of performing a vision zone assessment test (1210) can also include observing the patient's eye movements in response to a moving visual stimulus. The vision zone assessment test can be an optometric test.

The step of providing an appliance (1220) can include generating a lens having the non-transparent optical characteristic in an area registered with the patient's field area. Generating a lens can include applying an optical material to the lens. The optical material is selected from a group including inks, films, tapes, diffusants, and masks. The identified field areas can be different for the patient's right and left eyes. The field areas can be different between a right side and a left side of the patient's field of view.

While current embodiments of quadrantanopial occlusive lenses and a method of use have been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A therapeutic method of improving neurological performance of a patient comprising the steps:
performing a vision zone assessment test to identify a field area of a patient's field of view having a selected characteristic;
based on the identified field area, providing an appliance having a non-transparent optical characteristic associated with the field area;
the patient wearing the appliance; and
while the patient is wearing the appliance, the patient performing a selected activity associated with the neurological performance.

2. The therapeutic method of claim 1 wherein the neurological performance is associated with the quality of performance of the selected activity.

3. The therapeutic method of claim 1 wherein the selected activity is a physical activity.

4. The therapeutic method of claim 1 wherein performing a vision zone assessment test includes moving a visual target about the patient's field of view.

5. The therapeutic method of claim 1 wherein performing a vision zone assessment test includes observing the patient's eye movements in response to a moving visual stimulus.

6. The therapeutic method of claim 1 wherein the selected characteristic is abnormal eye movement when directed to a selected direction.

7. The therapeutic method of claim 1 wherein the non-transparent optical characteristic is opacity.

8. The therapeutic method of claim 1 wherein the non-transparent optical characteristic is translucency.

9. The therapeutic method of claim 1 wherein the non-transparent optical characteristic is reduced transmissivity.

10. The therapeutic method of claim 1 wherein the appliance is eyewear having a frame and a lens.

11. The therapeutic method of claim 1 wherein providing the appliance includes generating a lens having the non-transparent optical characteristic in an area registered with the patient's field area.

12. The therapeutic method of claim 1 wherein generating a lens includes applying an optical material to the lens.

13. The therapeutic method of claim 12 wherein the optical material is selected from a group including inks, films, tapes, diffusants, and masks.

14. The therapeutic method of claim 1 wherein the vision zone assessment test is an optometric test.

15. The therapeutic method of claim 1 wherein the selected activity is an athletic activity.

16. The therapeutic method of claim 1 wherein the field areas are different for the patient's right and left eyes.

17. The therapeutic method of claim 1 wherein the field areas are different between a right side and left side of the patient's field of view.
